(19) United States
(12) Patent Application Publication
(10) Pub. No.: US 2010/0331760 A1
(43) Pub. Date: Dec. 30, 2010

(54) BIODEGRADABLE IMPLANT FOR TREATING OR PREVENTING REPERFUSION INJURY

(75) Inventors: Liliana ATANASOSKA, Edina, MN (US); Aiden FLANAGAN, Galway (IE); Kent HARRISON, Maple Grove, MN (US); Liza DAVIS, St. Michael, MN (US); David SOGARD, Edina, MN (US)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 12/826,043

(22) Filed: Jun. 29, 2010

Related U.S. Application Data
(60) Provisional application No. 61/222,018, filed on Jun. 30, 2009.

(51) Int. Cl. A61N 1/30

(52) U.S. Cl. 604/20

(57) ABSTRACT

A biodegradable implant for iontophoretic therapeutic agent delivery is provided. The implant comprises a biodegradable battery and at least one biodegradable iontophoresis electrode assembly. The biodegradable battery comprises first and second biodegradable electrodes and a biodegradable polymer electrolyte layer between the first and second biodegradable electrodes. The biodegradable iontophoresis electrode assembly comprises a biodegradable iontophoresis electrode and a charged therapeutic agent. When the biodegradable battery is electrically connected to generate an electric current through the biodegradable iontophoresis electrode assembly, the charged therapeutic agent is delivered by iontophoresis to a target location. The charged therapeutic agent may be a scavenger for reactive oxygen species. The electrical connection of the biodegradable battery may be controlled remotely.

### CROSS REFERENCE TO RELATED APPLICATION

The present application claims priority to United States provisional application Ser. No. 61/222,018 filed Jun. 30, 2009, the disclosure of which is incorporated herein by reference in its entirety.

### TECHNICAL FIELD

The present disclosure relates to medical devices and methods for delivering a therapeutic agent to a target location. For example, the medical devices and method of the present disclosure may be used for preventing or reducing reperfusion injury.

### BACKGROUND

Blockage of arteries, such as the coronary arteries, can result in reduced blood flow to the downstream tissue, such as the heart muscle. In the case of the coronary arteries, such a blockage can lead to acute myocardial infarction or heart attack. Various treatments have been proposed to restore blood flow to the affected area, e.g., the ischemic myocardium. This process of restoring blood flow to the affected area is known as reperfusion.

Treatments that have been proposed to restore blood flow include thrombolytic therapy, percutaneous coronary intervention (PCI) and bypass surgery. Thrombolytic therapy involves the administration of therapeutic agents to open the blockage. Some thrombolytic agents that have been proposed or used include streptokinase, urokinase, and alteplase (recombinant tissue plasminogen activator, rtPA).

Percutaneous coronary intervention involves delivering a treatment device to the affected area of the blood vessel to open the blocked site. Commonly, an angioplasty procedure is performed in which a balloon catheter is tracked through the vasculature, and, once the balloon is at the constriction, the balloon is expanded to open the blockage. Often a stent is expanded and left at the site to help maintain the patency of the vessel.

Coronary artery bypass surgery involves a graft vessel being taken from the patient and implanted to bypass the area of blockage. Blood then is allowed to flow around the blockage through the bypass graft.

Reperfusion of blood flow to the ischemic tissue, while beneficial, can at times result in damage to the tissue. Because the affected tissue has been deprived of oxygen and nutrients, the restoration of blood flow can result in inflammation and oxidative damage. This is known as reperfusion injury.

Some techniques have been proposed to prevent or reduce reperfusion injury. For example, glisodin has been proposed as a therapeutic treatment. However, there continues to be a need for improved techniques to prevent or treat reperfusion injury.

### SUMMARY

In one embodiment, the present disclosure provides a biodegradable implant for iontophoretic therapeutic agent delivery, comprising a biodegradable battery and at least one biodegradable iontophoresis electrode assembly. The biodegradable battery comprises a first biodegradable electrode, a second biodegradable electrode, and a biodegradable polymer electrolyte layer between the first biodegradable electrode and the second biodegradable electrode. The biodegradable iontophoresis electrode assembly comprises a biodegradable iontophoresis electrode and a charged therapeutic agent. When the biodegradable battery is electrically connected to generate an electric current through the biodegradable iontophoresis electrode, the charged therapeutic agent is delivered by iontophoresis to a target location.

The biodegradable iontophoresis electrode assembly may comprise a therapeutic agent reservoir comprising a biodegradable polymer for containing the charged therapeutic agent. The biodegradable polymer may comprise, for example, poly(lactic-co-glycolic acid) (PLGA). The biodegradable iontophoresis electrode may be made of a suitable material, for example iron, magnesium, or alloys thereof. A surface of the iontophoresis electrode may be provided with a rice grain structure obtained by treating the surface with galvanic square waves.

The charged therapeutic agent may be a scavenger for reactive oxygen species. The charged therapeutic agent may be, for example, cationic methylene blue or ascorbate anion.

In some embodiments, the biodegradable implant may comprise an adsorbed, self-assembled layer of a therapeutic agent. The absorbed, self-assembled layer may comprise, for example, dimethylthiourea.

The first biodegradable electrode of the biodegradable battery may comprise, for example, iron or an iron alloy. The second biodegradable electrode of the biodegradable battery may comprise, for example, magnesium or a magnesium alloy.

The biodegradable polymer electrolyte layer of the biodegradable battery may comprise a biodegradable polymer such as poly(ethylene oxide) (PEO) or its derivatives, poly(lactic-co-glycolic acid) (PLGA), poly-ε-caprolactone (PCL), a polysaccharide, a cyanoethylpullulan polymer, collagen, or a combination thereof. The biodegradable polymer electrolyte layer may further comprise a salt. For example, the salt may be MgCl₂, CaCl₂ or FeCl₃. The biodegradable implant may further comprise a cathode intercalation layer between the first biodegradable electrode and the second biodegradable electrode.

In other embodiments, the present disclosure is directed to a method of providing iontophoretic therapeutic agent delivery by providing a biodegradable implant comprising at least one biodegradable iontophoresis electrode assembly and a biodegradable battery and electrically connecting the biodegradable battery to deliver a current through the biodegradable iontophoresis electrode assembly. The biodegradable battery has first and second biodegradable electrodes and a biodegradable polymer electrolyte layer between the first and second biodegradable electrodes. The biodegradable iontophoresis electrode assembly has a biodegradable iontophoresis electrode and a charged therapeutic agent. By electrically connecting the biodegradable battery to generate an electrical current through the iontophoresis electrode assembly, the therapeutic agent is delivered by iontophoresis to a target location. The step of electrically connecting the biodegradable battery may be done by remotely controlling a suitable connection.

In other embodiments, the present disclosure provides a method of preventing or reducing reperfusion injury in a subject by administering to the subject a biodegradable implant for iontophoretic therapeutic agent delivery. The biodegradable implant comprises a biodegradable battery and at least one biodegradable iontophoresis electrode assembly. The biodegradable battery comprises a first biodegradable electrode, a second biodegradable electrode; and a biodegradable polymer electrolyte layer between the first biodegradable electrode and the second biodegradable electrode. The biodegradable iontophoresis electrode assembly comprises a biodegradable iontophoresis electrode and a charged therapeutic agent. The method includes selectively electrically connecting the biodegradable battery such that the biodegradable battery generates an electrical current through the biodegradable iontophoresis electrode assembly which causes the charged therapeutic agent to be delivered by iontophoresis. In a first condition, the biodegradable battery does not supply current through the biodegradable iontophoresis electrode. The step of selectively electrically connecting the biodegradable battery to supply current through the biodegradable iontophoresis electrode can be accomplished by remotely controlling a connection to the biodegradable battery.

### BRIEF DESCRIPTION OF THE DRAWINGS

FIG. 1 shows a biodegradable implant for iontophoretic therapeutic agent delivery in accordance with one embodiment of the invention.

FIG. 2 shows a surface of an iron electrode having a rice grain structure obtained by treating the surface with galvanic square waves in accordance with one embodiment of the invention.

FIG. 3 shows the principle of operation of a biodegradable battery in accordance with one embodiment of the invention.

### DETAILED DESCRIPTION

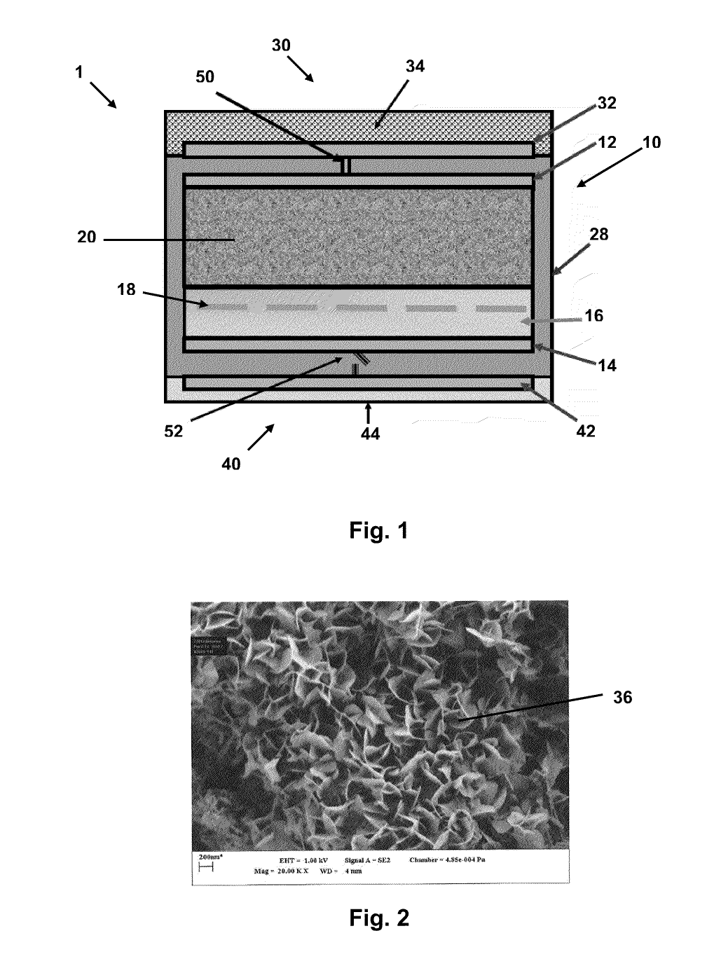

In accordance with one embodiment, FIG. 1 shows a biodegradable implant 1 for iontophoretic therapeutic agent delivery. The biodegradable implant 1 comprises a biodegradable battery 10 comprising a first biodegradable electrode 12, a second biodegradable electrode 14, and a biodegradable polymer electrolyte layer 16 between the first biodegradable electrode 12 and the second biodegradable electrode 14. A separator 18 is located between the between the first biodegradable electrode 12 and the second biodegradable electrode 14 in the biodegradable polymer electrolyte layer 16 of the biodegradable battery 10. The biodegradable battery 10 shown in FIG. 1 further comprises a cathode intercalation layer 20 between the first biodegradable electrode 12 and the second biodegradable electrode 14.

The biodegradable implant 1 shown in FIG. 1 further comprises a first biodegradable therapeutic agent delivery assembly 30 in the form of a biodegradable iontophoresis electrode assembly 30 comprising a biodegradable iontophoresis electrode 32 and a therapeutic agent reservoir 34. The therapeutic agent reservoir 34 comprises a biodegradable polymer containing a charged therapeutic agent. The embodiment in FIG. 1 also has a second biodegradable therapeutic agent delivery assembly 40 comprising a biodegradable electrode 42 and a layer 44 comprising a therapeutic agent. In this embodiment, the layer 44 is an adsorbed, self-assembled layer 44 of a therapeutic agent on the biodegradable electrode 42. In other embodiments, the second biodegradable therapeutic agent delivery assembly 40 is a second biodegradable iontophoresis electrode assembly, wherein the biodegradable electrode 42 is a biodegradable iontophoresis electrode 42 and the layer 44 comprises a therapeutic agent reservoir carrying a therapeutic agent that is oppositely charged from the therapeutic agent carried by therapeutic agent reservoir 34.

The biodegradable implant 1 shown in FIG. 1 has an insulating layer 28 of a biodegradable polymer that provides a barrier to an electrical circuit connection between the first biodegradable electrode 12 and the second biodegradable electrode 14, as described in further detail below. In the illustrated embodiment, the biodegradable implant 1 has an electrical contact 50 for electrically connecting the first biodegradable electrode 12 to the biodegradable iontophoresis electrode 32 and an electrical contact 52 for electrically connecting the second biodegradable electrode 14 to the biodegradable electrode 42. As depicted, the electrical contact 50 is closed so as to provide an electrical connection between the first biodegradable electrode 12 and the biodegradable iontophoresis electrode 32, and the electrical contact 52 is open so that there is no electrical connection between the second biodegradable electrode 14 and the biodegradable electrode 42. When the electrical contact 52 is closed, a circuit pathway is formed between the first biodegradable electrode 12 and the second biodegradable electrode 14. That is, a circuit pathway is formed from the second biodegradable electrode 14, through the electrical contact 52, through the biodegradable therapeutic agent delivery assembly 40, through the body tissue and/or biological fluids at the implantation site, around to the biodegradable iontophoresis electrode assembly 30, through the electrical contact 50, and to the first biodegradable electrode 12. When the electrical contact 52 is open, this circuit pathway is broken such that current does not flow. As will be described further below, the electrical contact 52 can be remotely actuated to close and form the electrical circuit as described. When the electrical circuit is closed and current passes, the therapeutic agent is delivered by iontophoresis from the biodegradable iontophoresis electrode assembly 30. When the layer 44 of the biodegradable therapeutic agent delivery assembly 40 is an adsorbed, self-assembled layer 44 of a therapeutic agent, such as dimethlythiourea, that therapeutic agent is delivered by reductive desorption (detachment). When the biodegradable therapeutic agent delivery assembly 40 is a second biodegradable iontophoresis electrode assembly, wherein the layer 44 comprises a therapeutic agent reservoir carrying a charged therapeutic agent, such as negatively charged ascorbate anions, that therapeutic agent is delivered by iontophoresis when the electrical circuit is closed and current passes.

Some example materials that may be employed in an embodiment as illustrated in FIG. 1 are as follows. In the biodegradable battery 10, the first biodegradable electrode 12 may serve as the cathode, and the second biodegradable electrode 14 may serve as the anode. A number of suitable biodegradable materials may be used to form the first and second biodegradable electrodes 12 and 14 of the biodegradable battery 10, as long as the electrodes are capable of forming an electrochemical cell that generates sufficient electric force to induce the release of the therapeutic agent by iontophoresis. For example, in certain embodiments, the first biodegradable electrode 12 may be made of, for example, iron or iron alloys, and the second biodegradable electrode 14 may be made of, for example, magnesium or magnesium alloys, calcium, zinc, or combinations thereof. In a biodegradable implant 1 for preventing or reducing reperfusion injury, magnesium can have therapeutic benefits since magnesium is known to reduce free oxygen radicals in an in vivo coronary occlusion-reperfusion model.

The biodegradable polymer electrolyte layer 16 may be formed of, for example, a biodegradable polymer such as poly(ethylene oxide) (PEO) or its derivatives, poly (lactic-co-glycolic acid) (PLGA), poly-ε-caprolactone (PCL), polysaccharides, a cyanoethylpullulan polymer, collagen, or combinations thereof. The biodegradable polymer electrolyte layer 16 shown in FIG. 1 may further comprise a salt. Any biodegradable salt that may render or assist in rendering the polymer electrolyte layer 16 ionically conductive may be used. The salt may be one that is present in the blood or tissue, such as MgCl₂, CaCl₂ or FeCl₃. Because MgCl₂ is a known scavenger of reactive oxygen species, MgCl₂ can have therapeutic benefits in a therapeutic agent delivery device for preventing or reducing reperfusion injury. The cathode intercalation layer 20 may comprise a suitable intercalation material such as, for example, Fe₂O₃ or FeₓPO₄OH.

The insulating layer 28 may comprise a biodegradable polymer suitable for insulation purposes in the desired application. Non-limiting examples of materials that may be used, depending on the application, include polycarboxylic acid, polyanhydrides, maleic anhydride polymers, polyorthoesters, poly-amino acids; polyethylene oxide, polyphosphazenes, polylactic acid, polyglycolic acid and copolymers and mixtures thereof such as poly(L-lactic acid) (PLLA), poly(D,L-lactide), poly(lactic acid-co-glycolic acid) (PLGA), 50/50 (DL-lactide-co-glycolide), polydioxanone, polypropylene fumarate, polydepsipeptides, polycaprolactone and co-polymers and mixtures thereof such as poly(D,L-lactide-co-caprolactone) and polycaprolactone co-butyl acrylate, polyhydroxybutyrate valerate and blends, polycarbonates such as tyrosine-derived polycarbonates and acrylates, polyiminocarbonates, and polydimethyltrimethylcarbonates, cyanoacrylate, calcium phosphates, polyglycosaminoglycans, macromolecules such as polysaccharides (including hyaluronic acid, cellulose, and hydroxypropyl methyl cellulose, gelatin, starches, dextrans, alginates and derivatives thereof), proteins and polypeptides, and mixtures and copolymers of any of the foregoing. The separator 18 may comprise any suitable biodegradable material.

The biodegradable iontophoresis electrode 32, which is electrically connected via electrical contact 50 to the first biodegradable electrode 12, may be made of, for example, iron or an iron alloy. The biodegradable electrode 42, which is selectively electrically connected via electrical contact 52 to the second biodegradable electrode 14, may be made of, for example, magnesium or a magnesium alloy, calcium, zinc, or a combination thereof

In the embodiment illustrated in FIG. 1, the therapeutic agent reservoir 34 of the biodegradable iontophoresis electrode assembly 30 is made of a suitable biodegradable polymer. Non-limiting examples of biodegradable polymers that may be used, depending on the application, include polycarboxylic acid, polyanhydrides, maleic anhydride polymers, polyorthoesters, poly-amino acids, polyethylene oxide, polyphosphazenes, polylactic acid, polyglycolic acid and copolymers and mixtures thereof such as poly(L-lactic acid) (PLLA), poly(D,L-lactide), poly(lactic acid-co-glycolic acid) (PLGA), 50/50 (DL-lactide-co-glycolide), polydioxanone, polypropylene fumarate, polydepsipeptides, polycaprolactone and co-polymers and mixtures thereof such as poly(D,L-lactide-co-caprolactone) and polycaprolactone co-butyl acrylate, polyhydroxybutyrate valerate and blends, polycarbonates such as tyrosine-derived polycarbonates and acrylates, polyiminocarbonates, and polydimethyltrimethylcarbonates; cyanoacrylate, calcium phosphates, polyglycosaminoglycans, macromolecules such as polysaccharides (including hyaluronic acid, cellulose, and hydroxypropyl methyl cellulose, gelatin, starches, dextrans, alginates and derivatives thereof), proteins and polypeptides, and mixtures and copolymers of any of the foregoing. In one example of the embodiment illustrated in FIG. 1 and described herein, the therapeutic agent reservoir 34 of the biodegradable iontophoresis electrode assembly 30 is made of poly(lactic-co-glycolic acid) (PLGA).

The charged therapeutic agent to be delivered by iontophoresis from the biodegradable iontophoresis electrode assembly 30, and the charged therapeutic agent to be delivered by iontophoresis from the biodegradable therapeutic agent delivery assembly 40 when that assembly is designed as a biodegradable iontophoresis electrode assembly 40, may comprise a therapeutic agent that is inherently charged or a therapeutic agent that is modified to bear a charge. For example, the charged therapeutic agent may comprise a therapeutic agent covalently attached to a charged molecule, a therapeutic agent that is non-covalently coupled to a charged molecule, a therapeutic agent that is attached to or encapsulated within a charged particle, or a combination thereof.

In certain embodiments, e.g., where the biodegradable implant 1 is a device for preventing or reducing reperfusion injury, the charged therapeutic agent to be delivered by iontophoresis from the biodegradable iontophoresis electrode assembly 30 and the charged therapeutic agent to be delivered by iontophoresis from the biodegradable therapeutic agent delivery assembly 40 when that assembly is designed as a biodegradable iontophoresis electrode assembly 40 may be scavengers for reactive oxygen species. In this way, once released, the therapeutic agents help prevent or reduce reperfusion injury.

In an embodiment such as that described above wherein the first biodegradable electrode 12 serves as the cathode of the biodegradable battery 10 and the second biodegradable electrode 14 serves as the anode of the biodegradable battery 10, the charged therapeutic agent that is to be delivered by iontophoresis from the biodegradable iontophoresis electrode assembly 30 will have a positive charge. In such an embodiment, if the biodegradable therapeutic agent delivery assembly 40 is a biodegradable iontophoresis electrode assembly 40, the charged therapeutic agent that is to be delivered by iontophoresis from the biodegradable iontophoresis electrode assembly 40 will have a negative charge.

In one example, the charged therapeutic agent of the biodegradable iontophoresis electrode assembly 30 is cationic methylene blue, contained in the therapeutic agent reservoir 34. As mentioned above, the biodegradable therapeutic agent delivery assembly 40 may comprise an adsorbed, self-assembled layer 44 of a therapeutic agent located on the biodegradable electrode 42. The layer 44 may be prepared by a number of suitable methods known in the art. For example, the chemical or electrochemical formation of an adsorbed, self-assembled layer of thiourea and substituted thiourea is described in A. E. Bolzan, et al., Electrochemical Study of Thiourea and Substituted Thiourea Adsorbates on Polycrystalline Platinum Electrodes in Aqueous Sulfuric Acid, Journal of Applied Electrochemistry 32: 611-620, which is incorporated herein by reference. In certain embodiments, the absorbed, self-assembled layer 44 may comprise dimethylthiourea, an effective scavenger of reactive oxygen species. Thus, in the example of FIG. 1, the therapeutic agent of the biodegradable therapeutic agent delivery assembly 40 is dimethylthiourea, which is located on the biodegradable electrode 42 as an absorbed, self-assembled layer 44 of dimethylthiourea. Other therapeutic agents may be used. If the biodegradable therapeutic agent delivery assembly 40 is a biodegradable iontophoresis electrode assembly 40, the charged therapeutic agent that is to be delivered by iontophoresis from the biodegradable iontophoresis electrode assembly 40 may be, for example, negatively charged ascorbate anion, and the layer 44 may comprise a therapeutic agent reservoir comprising a suitable biodegradable polymer such as one or more of the polymers described above in reference to therapeutic agent reservoir 34.

In certain embodiments, the therapeutic agent reservoir 34, and the layer 44 when it acts as a therapeutic agent reservoir, may comprise more than one charged therapeutic agent. For example, a cocktail of charged therapeutic agents may be contained in the therapeutic agent reservoir.

A therapeutic agent reservoir (such as therapeutic agent reservoir 34 or the layer 44 when it acts as a therapeutic agent reservoir) comprising a biodegradable polymer and a charged therapeutic agent may be formed in a number of suitable ways. For example, the charged therapeutic agent and a biodegradable polymer may be dissolved in an organic solvent and applied as a coating (such as by dip or spray coating) on the biodegradable electrode (e.g., biodegradable electrode 32 or 42), after which the solvent evaporates or is driven off. When the solvent evaporates or is driven off, the biodegradable electrode 32 or 42 is left with a biodegradable polymer coating containing the therapeutic agent.

In order to assist in adhesion of the biodegradable polymer to the respective biodegradable electrode 32 or 42, the surface of the electrode may be modified by treating the surface with galvanic square waves in order to achieve a rice grain structure. An image of the rice grain structure 36 is shown in FIG. 2. Similarly, the other electrodes may be treated to enhance adhesion, depending on the particular application. For example, the second biodegradable electrode 14 of the biodegradable battery 10 may be treated with galvanic square waves in order to achieve a rice grain structure to promote adhesion of the biodegradable polymer electrolyte layer 16.

An example of the use and operation of the embodiment illustrated in FIG. 1 with materials as described above is as follows. The biodegradable implant 1 is implanted in a patient at the location where the therapeutic treatment is desired, such as in or adjacent heart muscle in an area of reperfusion. The biodegradable implant may be implanted in a number of ways. For example, the implant may be delivered into and implanted in a heart chamber or placed on the outer surface of the heart in a region susceptible to reperfusion injury. The device may be delivered intravascularly or surgically. When implanted, the electrical contact 52 is open, such that current does not flow from the biodegradable battery 10 through the biodegradable electrodes 32 and 42.

At the desired time, a physician or other operator can selectively close the electrical contact 52 in order to cause the desired iontophoretic therapeutic agent delivery. The closing of the electrical contact 52 may be done by remote control. For example, a telemetry coil or means enabling remote operation as known in the art may be used. When the electrical contact 52 is closed, the biodegradable battery 10 is electrically connected to generate an electrical current through the biodegradable iontophoresis electrode assembly 30 and the biodegradable therapeutic agent delivery assembly 40 sufficient to cause the charged therapeutic agent to be delivered by iontophoresis from the biodegradable iontophoresis electrode assembly 30 (and also by iontophoresis from the biodegradable therapeutic agent delivery assembly 40 when it is designed as a biodegradable iontophoresis electrode assembly). That is, when the electrical contact 52 is closed, an electrical circuit is completed, allowing current flow. From the second biodegradable electrode 14, the circuit travels through the electrical contact 52, through the biodegradable therapeutic agent delivery assembly 40, through the body tissue and/or biological fluids at the implantation site, around to the biodegradable iontophoresis electrode assembly 30, through the electrical contact 50, and to the first biodegradable electrode 12.

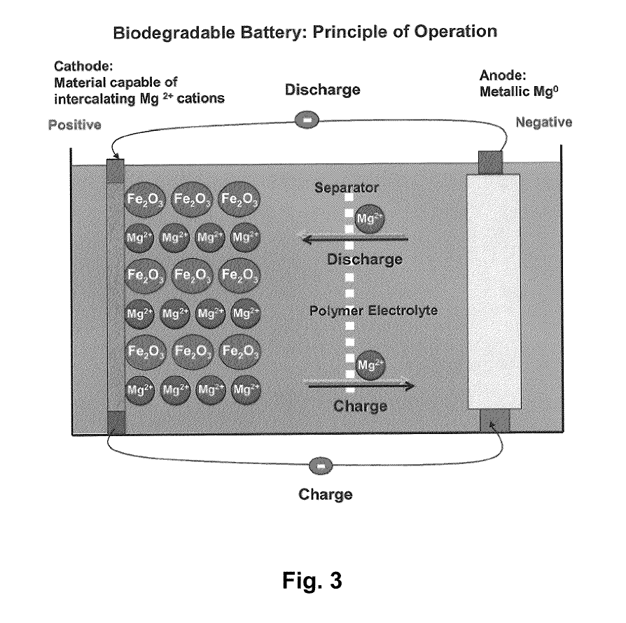

When the circuit is closed, the biodegradable battery 10 generates an electric force sufficient to cause the charged therapeutic agent to be delivered by iontophoresis from the biodegradable iontophoresis electrode assembly 30 (and also by iontophoresis from the biodegradable therapeutic agent delivery assembly 40 when it is designed as a biodegradable iontophoresis electrode assembly) and elute to a target location. FIG. 3 shows the principle of operation of a biodegradable battery 10 in accordance with one embodiment of the invention. The biodegradable battery comprises an iron cathode and a magnesium anode. In the discharge mode, Mg²⁺cations move toward the iron cathode through the polymer electrolyte inside the battery, while electrons move from the magnesium anode to the iron cathode outside the battery in the circuit as described above. In the embodiment illustrated in FIG. 3, the cathode intercalation layer comprises Fe₂O₃, but other suitable materials such as FeₓPO₄OH may be used. The electric current from the biodegradable battery 10 causes the biodegradable iontophoresis electrode 32 to become positively charged and the biodegradable electrode 42 to become negatively charged. This thereby causes the charged therapeutic agent at the biodegradable iontophoresis electrode assembly 30 (and the charged therapeutic agent at the biodegradable therapeutic agent delivery assembly 40 when it is designed as a biodegradable iontophoresis electrode assembly) to be delivered by iontophoresis. For example, a positively charged therapeutic agent such as cationic methylene blue will be delivered by iontophoresis from a positively charged biodegradable iontophoresis electrode 32. Similarly, a negatively charged therapeutic agent such as ascorbic anion will be delivered by iontophoresis from a negatively charged biodegradable iontophoresis electrode 42.

The components of the device may be selected so that the current generated does not damage tissue. For example, the current may be maintained at the micro ampere level to avoid tissue damage. In addition, the voltage may be kept low, for example not more than about 3 volts. The release rate of the charged therapeutic agents can be adjusted by changing the magnitude of the electrical current. An increase in the current results in a higher drug release rate.

As will be appreciated by persons of ordinary skill in the art from the description herein, a biodegradable implant for iontophoretic therapeutic agent delivery in accordance with the present disclosure may be used to treat or reduce reperfusion injury. Tissue ischemia and the decrease of oxygen intake by the cells result in accumulation of hypoxanthine and the conversion of xanthine dehydrogenase into xanthine oxidase. During reperfusion, the accumulated hypoxanthine is converted by the xanthine oxidase in the presence of molecular oxygen (O₂) into xanthine and free radicals of oxygen: superoxides, peroxides and hydroxyl. These reactive oxygen species cause an inflammatory process characterized by the increase in endothelial permeability to fluids, macromolecules, and inflammatory cells. Scavengers for reactive oxygen species, such as methylene blue, ascorbic acid, and ascorbic anion, suppress the production of free oxygen radicals by competing with O₂ for the electrons from xanthine oxidase, and thus may be used for preventing or reducing reperfusion injury.

A biodegradable implant in accordance with certain embodiments of the present disclosure can effectively prevent or reduce reperfusion injury caused by reactive oxygen species by the iontophoretic delivery of suitable therapeutic agents. Because the circuit connection is controllable, a biodegradable implant in accordance with certain embodiments of the disclosure can have both acute and prolonged effects. Thus, the device can be capable of delivering rapidly a therapeutic agent into myocardial cells to counter the burst of oxygen free radicals generated early in reperfusion, and also providing an extended release of a therapeutic agent and maintaining sufficient therapeutic agent levels to protect against low levels of reactive oxygen species produced during the subsequent hours.

In accordance with certain embodiments of the present disclosure, the release rate of the therapeutic agent can be altered by adjusting the current flow. The device may be designed to generate a large current flow initially to rapidly release a therapeutic agent (e.g., a scavenger for reactive oxygen species) into myocardial cells to counter the burst of oxygen free radicals in the initial hours of reperfusion. In the subsequent hours, the current flow may be maintained at relatively low levels to provide an extended release of a therapeutic agent to protect against low levels of reactive oxygen species. The levels of current can be controlled, for example, by having multiple batteries as described herein and selectively coupling a select number of individual batteries as desired at a given time. Therefore, the biodegradable battery arrangement provides a power source that enables both an initial burst and an extended release of a therapeutic agent to counter the reactive oxygen species during both stages of reperfusion injury.

As will be appreciated by persons of ordinary skill in the art from the description herein, a biodegradable implant for iontophoretic therapeutic agent delivery in accordance with the present disclosure can be made fully biodegradable when every component is biodegradable. Thus, the device can provide the additional advantage of avoiding the undesirable side effects (e.g., chronic inflammation) that may be associated with non-biodegradable materials that remain in the body for a long time.

A biodegradable implant in accordance with the present disclosure may be any suitable size. Also, the device may be any suitable shape, such as a patch, helix, ring, etc.

A biodegradable implant in accordance with the present disclosure may be implanted or otherwise used in body structures, cavities, or lumens such as the vasculature, gastrointestinal tract, abdomen, peritoneum, airways, esophagus, trachea, colon, rectum, biliary tract, urinary tract, prostate, brain, spine, lung, liver, heart, skeletal muscle, kidney, bladder, intestines, stomach, pancreas, ovary, uterus, cartilage, eye, bone, joints, and the like.

While certain therapeutic agents deliverable by a device as described herein have been set forth above, other therapeutic agents may be used depending on the desired application. Exemplary non-genetic therapeutic agents include anti-thrombogenic agents such heparin, heparin derivatives, prostaglandin (including micellar prostaglandin El), urokinase, and PPack (dextrophenylalanine proline arginine chloromethylketone); anti-proliferative agents or anti-restenosis agents such as enoxaparin, angiopeptin, paclitaxel, sirolimus (rapamycin), tacrolimus, everolimus, zotarolimus, biolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl) ethyleneglycol-N,N,N′,N′-tetraacetic acid and mixtures thereof; antibiotics such as gentamycin, rifampin, minocyclin, and ciprofloxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; inhibitors of heat shock proteins such as geldanamycin; angiotensin converting enzyme (ACE) inhibitors; beta-blockers; βAR kinase (βARK) inhibitors; phospholamban inhibitors; protein-bound particle drugs such as ABRAXANE™; structural protein (e.g., collagen) cross-link breakers such as alagebrium (ALT-711); and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (MCP-1) and bone morphogenic proteins (“BMP's”) such as, for example, BMP-2,BMP-3, BMP-4, BMP-5, BMP-6 (VGR-1), BMP-7 (OP-1), BMP-8, BMP-9, BMP-10, BMP-11, BMP-12, BMP-13, BMP-14 or BMP-15. Preferred BMP's are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factors α and β, platelet-derived endothelial growth factor, platelet-derived growth factor, tumor necrosis factor α, hepatocyte growth factor, and insulin-like growth factor. A non-limiting example of a cell cycle inhibitor is a cathespin D (CD) inhibitor. Non-limiting examples of anti-restenosis agents include p15, p16, p18, p19, p21, p2′7, p53, p57, Rb, nFkB and E2F decoys, thymidine kinase and combinations thereof, and other agents useful for interfering with cell proliferation.

Exemplary small molecules include hormones, nucleotides, amino acids, sugars, and lipids and compounds having a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or can be genetically engineered. Non-limiting examples of cells include side population (SP) cells, lineage negative (Lin⁻) cells including Lin⁻CD34⁻, Lin⁻CD34⁺, Lin⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells. Any of the therapeutic agents may be combined to the extent such combination is biologically compatible.

The foregoing description and examples have been set forth merely to illustrate the invention and are not intended to be limiting. Each of the disclosed aspects and embodiments of the present invention may be considered individually or in combination with other aspects, embodiments, and variations of the invention. Modifications of the disclosed embodiments incorporating the spirit and substance of the invention may occur to persons skilled in the art and such modifications are within the scope of the present invention.

What is claimed is:

1. A biodegradable implant for iontophoretic therapeutic agent delivery, comprising:
a biodegradable battery, comprising:
a first biodegradable electrode;
a second biodegradable electrode; and
a biodegradable polymer electrolyte layer between the first biodegradable electrode and the second biodegradable electrode; and
at least one biodegradable iontophoresis electrode assembly comprising a biodegradable iontophoresis electrode and a charged therapeutic agent;
wherein when the biodegradable battery is electrically connected to generate an electric current through the biodegradable iontophoresis electrode assembly, the charged therapeutic agent is delivered by iontophoresis to a target location.

2. The biodegradable implant according to claim 1, wherein the biodegradable iontophoresis electrode assembly further comprises a therapeutic agent reservoir comprising a biodegradable polymer for containing the charged therapeutic agent.

3. The biodegradable implant according to claim 2, wherein the biodegradable polymer comprises poly(lactic-co-glycolic acid) (PLGA).

4. The biodegradable implant according to claim 1, wherein the biodegradable iontophoresis electrode is selected from a group consisting of iron, magnesium, and alloys thereof.

5. The biodegradable implant according to claim 4, wherein a surface of the iontophoresis electrode has a rice grain structure obtained by treating the surface with galvanic square waves.

6. The biodegradable implant according to claim 1, wherein the charged therapeutic agent is a scavenger for reactive oxygen species.

7. The biodegradable implant according to claim 1, wherein the charged therapeutic agent is cationic methylene blue.

8. The biodegradable implant according to claim 1, wherein the charged therapeutic agent is ascorbate anion.

9. The biodegradable implant according to claim 1, wherein the biodegradable implant comprises an adsorbed, self-assembled layer of therapeutic agent.

10. The biodegradable implant according to claim 9, wherein the adsorbed, self-assembled layer comprises dimethylthiourea.

11. The biodegradable implant according to claim 1, wherein the first biodegradable electrode of the biodegradable battery comprises iron or an iron alloy.

12. The biodegradable implant according to claim 1, wherein the second biodegradable electrode of the biodegradable battery comprises magnesium or a magnesium alloy.

13. The biodegradable implant according to claim 1, wherein the biodegradable polymer electrolyte layer comprises a biodegradable polymer selected from the group consisting of poly(ethylene oxide) (PEO) and its derivatives, poly(lactic-co-glycolic acid) (PLGA), poly-ε-caprolactone (PCL), polysaccharides, a cyanoethylpullulan polymer, collagen, and combinations thereof.

14. The biodegradable implant according to claim 1, wherein the biodegradable polymer electrolyte layer further comprises a salt.

15. The biodegradable implant according to claim 14, wherein the salt is selected from a group consisting of MgCl₂, CaCl₂ and FeCl₃.

16. The biodegradable implant according to claim 1, wherein the biodegradable battery further comprises a cathode intercalation layer between the first biodegradable electrode and the second biodegradable electrode.

17. A method of providing iontophoretic therapeutic agent delivery, comprising:
providing a biodegradable implant comprising at least one biodegradable iontophoresis electrode assembly having a biodegradable iontophoresis electrode and a charged therapeutic agent and a biodegradable battery having first and second biodegradable electrodes and a biodegradable polymer electrolyte layer between the first and second biodegradable electrodes;
and electrically connecting the biodegradable battery to generate an electrical current through the biodegradable iontophoresis electrode assembly sufficient to cause the charged therapeutic agent to be delivered by iontophoresis to a target location.

18. The method according to claim 17, wherein the step of electrically connecting the biodegradable battery is done by remotely controlling a connection to the biodegradable battery.

19. A method of preventing or reducing reperfusion injury in a subject, comprising:
implanting in a subject a biodegradable implant for iontophoretic therapeutic agent delivery, the biodegradable implant comprising:
a biodegradable battery, comprising:
a first biodegradable electrode;
a second biodegradable electrode; and
a biodegradable polymer electrolyte layer between the first biodegradable electrode and the second biodegradable electrode; and
at least one biodegradable iontophoresis electrode assembly comprising a biodegradable iontophoresis electrode and a charged therapeutic agent; and
selectively electrically connecting the biodegradable battery to generate an electrical current through the biodegradable iontophoresis electrode assembly sufficient to cause the charged therapeutic agent to be delivered by iontophoresis to a target location.

20. The method according to claim 19, wherein in a first condition, the biodegradable battery does not supply current through the biodegradable iontophoresis electrode, and wherein the step of selectively electrically connecting the biodegradable battery is done by remotely controlling a connection to the biodegradable battery.
